(19) United States
(12) Patent Application Publication
(10) Pub. No.: US 2011/0306859 A1
(43) Pub. Date: Dec. 15, 2011

(54) MULTIPURPOSE, MODULAR PLATFORM FOR MOBILE MEDICAL INSTRUMENTATION

(76) Inventors: Enrique SALDIVAR, Santee, CA (US); Mehran Mehregany, San Diego, CA (US)

(21) Appl. No.: 13/102,817

(22) Filed: May 6, 2011

Related U.S. Application Data
(60) Provisional application No. 61/332,024, filed on May 6, 2010.

(51) Int. Cl. A61B 5/145; A61B 5/01; A61N 5/06; A61B 5/0488; A61M 5/142; A61B 5/0402; A61B 5/08; G08B 1/08; A61N 1/362

(52) U.S. Cl. 600/365; 340/539.12; 600/549; 607/88; 607/9; 604/151; 600/509; 600/532; 600/546

(57) ABSTRACT

A system provides for mobile medical instrumentation for use with a body. The system includes a primary wireless communication device, such as a cell phone or tablet computer, and one or more cradles. The cradles provide an interface to the body which provides for input of signals from the body, and optionally, therapeutic outputs to the body. The cradle and the primary wireless communication device may be releasably joined together so as to form a unitary structure when contacting the body. Alternately, the cradle may interface with the body, and communicate wirelessly to the primary wireless communication device. A cradle adapted to provide for a portable electrocardiogram includes a plurality of electrodes adapted for contact or non-contact sensing of the body. In the preferred embodiment, three electrodes are arranged in a triangular arrangement, most preferably a Einthoven triangular arrangement, with the distance between electrodes being 4 centimeters or less.

### RELATED APPLICATION STATEMENT

This application claims priority to and benefit of U.S. Provisional Application Ser. No. 61/332,024, filed May 6, 2010, entitled “Multipurpose, Modular Platform for Mobile Medical Instrumentation”, the content of which is hereby incorporated by reference in its entirety as if fully set forth herein.

### FIELD OF THE INVENTION

The present inventions relate to medical instrumentation systems. More particularly, they relate to multipurpose, modular platforms for mobile medical instrumentation.

### BACKGROUND OF THE INVENTION

There are four primary problems solved with this invention. The first is the issue of cost or price of medical equipment. Generally, medical equipment used in hospitals is expensive. One of the reasons for the excessive pricing is the complicated hardware and electronics. A common philosophy in design is to implement the microprocessing data processing units imbedded in the internal circuitry. This approach increases the research and development cycle, as the working load of the designing teams must accommodate the demand of the imbedded computer capabilities. This approach also increases substantially the cycles of modifications and improvements.

The second primary problem addressed is an issue of physical size. Medical equipment used in hospitals generally are large and voluminous. One of the reasons is the method used to process the data and the communication protocols. Another common philosophy in design is the direct connection to a personal computer to the medical device to perform the communication and computing duties. Although this solution partially remedies some of the costs in design, it adds volume and mass to the medical equipment.

The third issue addressed is one of flexibility of equipment and systems. Generally, medical equipment has a task-specific design. This narrow functionality has a direct impact on the total cost that an institution spends on medical equipment, as currently it is necessary to buy several pieces of expensive equipment to complete several medical tasks.

The fourth issue addressed is one of providing electrical connection between the equipment and the patient or user. Generally, medical equipment that captures biomedical signals (for example, electrocardiographs) connects the electrodes on the patient through a cumbersome system of wires. Additionally, the wires are affixed to the skin with glue. This feature limits the use of the system (for example, in pediatrics medicine and neonatal care, as peeling of the glued electrodes from the skin of the infant damages their delicate skin) or creates patient discomfort (for example, peeling of glued electrodes from hairy skins in adults).

Various systems have sought to address one or all of these issues. No optimal solution has yet been presented.

The Abbott i-STAT Point-of-Care system utilizes a flexible platform, based on disposable cartridges. This method allows the operator to perform different laboratory tests by simply selecting the proper cartridge for the test. In addition, the i-STAT is a handheld portable system. Thus, this system is a solution for large size in medical equipment. This system offers flexibility as it can be used to perform different biochemical tests with the same basic equipment by simply changing the cartridge.

The i-STAT system presents several problems. First, it performs only biochemical measurements. Thus, the system is not flexible enough to be adopted as a general platform for medical diagnosis. Second, the i-STAT system utilizes a proprietary computing and data processing platform, increasing the price and cost of development, as it is a purpose-specific solution. Third, the i-STAT system is not wireless and requires, either, manual data entry or connection to a computer via cable to access the data.

The Kiwok BodyKom system is an electrocardiograph system with a set of wires that connects the electrodes to a wireless unit connected to a cell phone via Bluetooth (http://www.kiwok.se/index.php). This system is a solution to large size and high price problems in medical equipment.

The deficiencies of the BodyKom system are as follows. First, it is exclusive for electrocardiography. Thus, the system is not flexible enough to be adopted as a general platform for medical diagnosis. Second, the BodyKom presents the problem of having wires for the connection of the electrodes, which limits the usability as the system is cumbersome. The presence of cables hinders the rapid readout of the electrocardiogram, presenting a critical problem in emergency situations. Third, the system presents the problem of utilizing glue to fix the electrodes to the skin. This feature limits the use of the system in pediatrics medicine and neonatal care.

The DRE system is a complete electrocardiogram. The system is small and portable, capable of connecting directly to a personal computer. The DRE system has wires to connect to the electrodes. This system is a solution to the problem of large size in medical equipment.

The DRE system presents several problems. First, the DRE is exclusive for electrocardiography. Thus, the system is not flexible enough to be adopted as a general platform for medical diagnosis. Second, the DRE system presents the problem of having wires for the connection of the electrodes, which limits the usability as the system is cumbersome. The presence of cables hinders the rapid readout of the electrocardiogram, presenting a critical problem in emergency situations. Third, the DRE system presents the problem of utilizing glue to fix the electrodes to the skin. This feature limits the use of the system in pediatrics medicine and neonatal care. Fourth, the DRE system utilizes a proprietary data display and processing platform, increasing the price and cost of development, as it is a purpose-specific solution.

Various groups have attempted solutions which include wireless EKG monitoring systems. A survey of various efforts may be found in the article “Development and Evaluation of a Bluetooth EKG Monitoring Sensor”, Proulx, J., Clifford, R., Sorensen, S., Dah-Jye, Lee, Archibald, J., Dept. of ECEn, Brigham Young Univ., Provo, Utah; (published in Computer-Based Medical Systems, 2006. CBMS 2006. 19th IEEE International Symposium on Computer-Based Medical Systems, page(s): 507-511, Salt Lake City, Utah, ISSN: 1063-7125, ISBN: 0-7695-2517-1, INSPEC Accession Number: 9187352, Digital Object Identifier: 10.1109/CBMS.2006.74, Current Version Published: 2006 Jul. 5). In the system developed and evaluated by the authors, a 3 lead wired system is connected to the patient in the classic Einthoven Triangle configuration, with the wires connected to an EKG sensor. An Analog-to-digital converter then passes the EKG signal to a serial-toBluetooth module. Bluetooth communication is than transmitted to a cellular phone, on which the EKG data is stored and/or displayed. Optionally, the data is then transmitted from the phone to a remote location, such as for analysis by medical professionals.

Despite the desirability for a solution maximizing the desirable advantages discussed herein, no solution has yet to be presented.

### BRIEF SUMMARY OF THE INVENTION

The invention is an adaptable multi-purpose medical instrumentation platform that uses the computing capabilities, communications, display capabilities, and other functions of a cellular telephone. The invention utilizes a series of cradles capable of housing the cellular telephone. The cradles preferably contain all the electronics, sensors, and additional hardware necessary to function. Each cradle has one or more specific application purposes. The user places the cell phone inside the cradle that confers the device the desired functionality. For its use, the user simply selects the cradle and places the cellular telephone inside the cradle. The cellular telephone has a series of preprogrammed software applications that complete the functionality of the invention. Preferably, there is a specific application software program for each specific cradle.

In one aspect of the invention, a system is provided for mobile medical instrumentation for use with a body. The system includes a primary wireless primary communication device. In one preferred embodiment, the wireless primary communication device is a cellular telephone. That device, in turn, preferably includes a housing, a display adjacent at least a portion of the housing, an internal processor, a wireless external communication circuit, a power source, and a communication input/output port adapted for communication with a cradle. The cradle, in turn, preferably includes an input sensor for receiving input from the body, the input sensor generating an output signal corresponding to the input, a processing circuit for receiving and processing the input sensor output signal, and a cradle input/output port adapted for communication with the wireless primary communication device and with the processing circuit.

In yet another aspect of the invention, the system provides for mobile medical instrumentation for use with a body. The system includes a primary wireless communication device, such as a cell phone or table computer, and one or more cradles. The cradles provides and interface to the body which provides for input of signals from the body, and optionally, therapeutic outputs to the body. Communication input/output ports provide for communication between the primary wireless communication device and the cradle. The cradle and the primary wireless communication device may be releasably joined together so as to form a unitary structure when contacting the body. Alternately, the cradle may interface with the body, and communicate wirelessly to the primary wireless communication device. A cradle adapted to provide for a portable electrocardiogram includes a plurality of electrodes adapted for contact or non-contact sensing of the body. In the preferred embodiment, three electrodes are arranged in a triangular arrangement, most preferably an Einthoven triangular arrangement, but with the distance between electrodes being 4 centimeters or less.

In yet another aspect, apparatus and methods include a cradle for a modular system for mobile medical instrumentation for use with a body, the cradle being adapted to interface with a wireless primary communication device. The cradle preferably includes an input sensor for receiving input from the body, the input sensor generating an output signal corresponding to the input, a processing circuit for receiving and processing the input sensor output signal, and a cradle input/output port adapted for communication with the wireless primary communication device and with the processing circuit.

In yet another aspect, the invention relates to a cradle for a modular system for mobile medical instrumentation for use with a body, adapted to measure the electrocardiogram of a patient, the cradle being adapted to interface with a wireless primary communication device, the wireless primary communications device having a face surface of length L and width W, an input sensor for receiving input from the body, the input sensor including multiple electrodes, the electrodes being located within the dimensions L and W, wherein L and W are sized to fit within the region of the size of the face surface, preferably where L and W are 4 centimeters or less, or 3 centimeters or less, or in from 1 to 2 centimeters. The system further preferably includes an input sensor generating an output signal corresponding to the input, a processing circuit for receiving and processing the input sensor output signal, and a cradle input/output port is adapted for communication with the wireless primary communication device and with the processing circuit.

In yet another aspect of the invention, a communications unit is provided for mobile medical communication for communications between two or more wireless units having incompatible wireless data communications formats. The communications unit provides for use with a first medical device adapted for use with a body and for wireless communication operating in a first wireless data format, and for use with a primary communication device, the primary communication device operating in a second wireless data format, the first wireless data format being incompatible with the second wireless data format. Preferably, the communications unit has a first receiver adapted to receive communications from the first medical device in a first wireless data format, a translator to convert a first wireless data format to a second wireless data format utilized by the wireless primary communication device, and a transmitter to send the converted second wireless data format to the primary communication device. Optionally, the primary communication device may operate with two or more wireless data formats, such as where the second wireless data format for communication with the communications unit is a local wireless data format, such as Bluetooth, and the other wireless data format is for cellular communications, such as GSM or CDMA.

The systems, apparatus and methods of this invention have numerous advantages compared to prior designs. First, they provide a universal platform that minimizes the development cycle and production cost in biomedical equipment. This is a unique feature not found in other devices. Second, they are more economical to implement than other systems as the present invention may utilize the computing hardware already existing in a cellular telephone. Third, the device of the invention is more economical to implement than other systems as the present invention may take advantage of the display capabilities already existing in a communication device, e.g., cellular telephone. Fourth, the device of the invention is more flexible than other systems. This flexibility allows it to perform as a universal diagnostics platform, with applications in biochemistry, pathology, hematology, medical imaging, and bio-medical signals, or others known to those skilled in the art. Fifth, in electrocardiography, the device of the invention allows the direct application of the electrodes to the skin of the patient avoiding the use of electrodes glued to the skin of the patient. Alternately, a contact-less, e.g. capacitive system, may be used to obtain ECG signals through clothing. These features allow rapid readings in emergency situations, and avoids potential damage to the skin of the patient (as in newborns, premature babies, and burned patients). Sixth, the device of the invention can be used in both diagnostics and therapeutics. Examples of therapeutics include phototherapy and electrotherapy. This is a unique feature not found in other diagnostics devices.

### BRIEF DESCRIPTION OF THE DRAWINGS

FIGS. 1 A and B are diagrams of the device of the invention. These figures are a general representation of the device. FIG. 1A shows the top view of the cradle 10, with a cellular telephone 40 inside. FIG. 1B shows a lateral section 44 of the device of the invention with the cellular telephone 40 inside the cradle 10. The electronics and other hardware are encased in the cradle 10. The cradle 10 contains the necessary hardware for the input 12 and output 24 of signals of biomedical relevance.

FIGS. 2 A and B show a graphic representation of the placement of the electrodes on the body 60 for electrocardiographic recording. FIG. 2A shows a typical, prior art, electrode placement in bipolar leads, forming the Einthoven's triangle. See 62, 64, and 66. FIG. 2B shows the distribution of the electrodes in an Einthoven's triangle 68 with reduced dimensions of this invention.

FIG. 3 is a general configuration of the device of the invention housing an electrocardiograph (ECG). The left top panel shows the device in operation with the cellular telephone 40 inside the cradle 10. The two triangular markers show the level of the cross section shown in the middle left panel of cradle 10 in cross-section. The middle left panel shows the cross section of the cradle without the cellular telephone inside. The lower left panel shows the back side of the cradle with the three electrodes 14. The right panel shows the device in operation and its placement on the chest of the patient. The electrocardiogram is directly shown on the screen 42 of the cellular telephone 40.

FIGS. 4 A, B and C show the connectivity of the electrodes 14 in the invention housing an electrocardiograph. FIG. 4A shows the electrodes 14 and their relative placement. FIG. 4B shows the connectivity of the electrodes 14 with the interior of the cradle 10. FIG. 4C shows the connectivity of the electrodes to the differential amplifier circuit including operational amplifiers 68 in each one of the bipolar leads: I (top), II, (middle), and III (bottom).

FIG. 5 is a graphic representation of the operation of the device, housing a low power transmitter/receptor 70 of biomedical signals. The left panel shows a handheld device 70 with three electrodes for electrocardiography (similar to the configuration shown in FIGS. 2, 3, and 4). This handheld device 70 has a low power transmitter that broadcasts the biomedical signal 72. The top right panel shows the device of the invention with the low power transmitter/receiver 70 encased in the cradle 10. The right lower panel shows the usage of the handheld device 70, hand-placed on the chest of the patient.

FIG. 6 is a graphic representation of the operation of the device, housing a low power transmitter/receptor of biomedical signals. The transmitter in this figure is mounted on an adhesive patch 74, which has electrodes in a configuration similar to the one described in FIGS. 2, 3, 4, and 5. This adhesive patch has a low power transmitter that sends the biomedical signal to the low power receiver encased in the cradle 10.

FIGS. 7 A, B, C and D are graphic representations of the device of the invention housing point-of-care measurements, such as amperometric measurements using substrate specific enzyme-linked reactions or volt-metric measurements. The cradle 10 has a lateral cartridge slot 80 for the insertion of the sample. FIG. 7A shows the cellular telephone 40 inside the cradle 10 and the middle top panel shows a lateral view of the cradle 10 with the cartridge slot 80. FIG. 7B shows the cartridge, with the contact electrodes 88. FIGS. 7 C and D show the loading of the cartridge with the biological sample and the insertion of the cartridge inside the cradle 10 for measurements, respectively.

FIG. 8 is a schematic block diagram of the system including a primary wireless communications device, such as a cell phone 40, and a cradle 10.

### DETAILED DESCRIPTION OF THE INVENTION

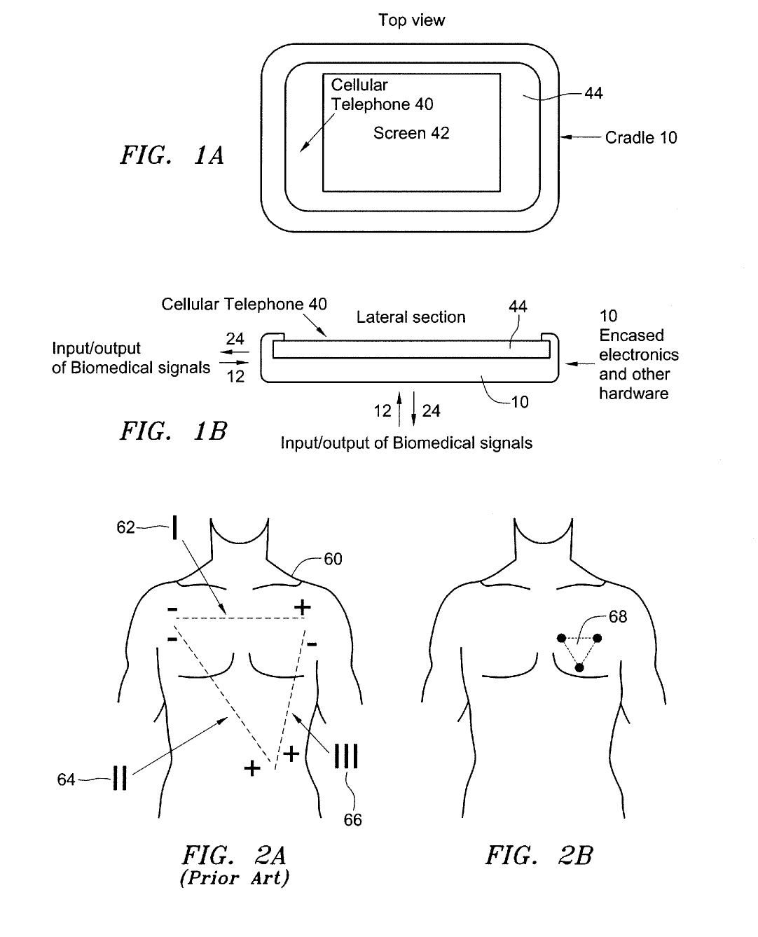

FIGS. 1 and 8 show the general configuration of the device of the invention. FIGS. 1 A and B are diagrams of the device of the invention. These figures are a general representation of the device. FIG. 1A shows the top view of the cradle 10, with a cellular telephone 40 inside. FIG. 1B shows a lateral section 44 of the device of the invention with the cellular telephone 40 inside the cradle 10. The electronics and other hardware are encased in the cradle 10. The cradle 10 contains the necessary hardware for the input 12 and output 24 of signals of biomedical relevance. FIG. 8 is a schematic block diagram of the system including a primary wireless communications device, such as a cell phone 40, and a cradle 10.

The device of the invention uses the computer capabilities of a cellular telephone for different medical applications. The invention uses the display capabilities of a cellular telephone to communicate data to the user. The invention uses the transmission/receiver capabilities of a cellular telephone to send and receive biomedical information to other devices, including computers. The invention uses the input/output port of a cellular telephone to enter data, or biomedical signals, to the cellular telephone or other devices, including the hardware of the invention. The invention optionally uses the battery power of a cellular telephone to operate the hardware of the invention. The invention uses the transmitting/receiving capabilities of a cellular telephone to transmit/receive the biomedical data of the patient. The invention houses the electronic circuits and additional hardware in the body of a case that houses the cellular telephone.

The invention has a cellular telephone holder, referred to as the cradle 10. The device of the invention includes the necessary transducers 14, 22 and hardware encased in the cradle 10, for additional features which allows it to receive and transmit relevant biomedical signals. The input and output of the device of the invention can be used for diagnosis, treatment, patient identification, disease monitoring, patient evaluation, or any other activity related to medical clinical practice.

With particular reference to FIG. 1 A, the top panel depicts the top view of the cradle 10 housing the cellular telephone 40. FIG. 1B shows the lateral section of the device of the invention showing the placement of the cellular telephone inside the cradle 10. The cradle 10 can receive and send biomedical signals from any of its facets, including front, back, and lateral.

The device of the invention has an electronic docking station that allows electronic communication between the electronics and hardware of the cradle 10 and the cellular telephone. This connector also provides the electric power to operate the electronics and other hardware in the cradle 10 by allowing access to the power of the battery 54 of the cellular telephone.

The device of the invention preferably has several cradles 10, each one for a different medical application. Each cradle 10 contains purpose-specific electronics and other hardware for proper performance. Each cradle 10 utilizes a specific software application that allows the communication with the hardware of the cradle 10, proper data acquisition and transmission of the biomedical data, and other functions as required. In an example, these software applications can be recognized by the telephone depending on the cradle 10 utilized, the telephone automatically starts the appropriate software application just by being connected to a specific cradle 10. Further, additional application software (apps) may be downloaded to the cradle 10 and/or the wireless primary communication device, e.g., cellular telephone, as desired.

With particular reference to FIG. 8, the wireless primary communications device, such as a cellular telephone 40, is shown in combination with the cradle 10. The main electrical and mechanical components are shown in a block diagram format.

As to the cradle 10, it preferably includes an input sensor 14 for receiving input 12 from the body. The input sensor generates an output signal corresponding to the input. A processing circuit 38 receives and processes the input sensor output signal. Optionally, an amplifier 16 is adapted to receive an input from the sensor and the control system/processor 38. Preferably, the amplifier is a low noise amplifier. As to optional components, a digital to analog converter 16 may be disposed between the control system and the amplifier. Further, a digital signal processor may be disposed between the control system and the digital to analog converter. An analog to digital converter may be coupled to the amplifier. Finally, optionally, a digital signal processor may be coupled to the digital to analog converter.

A cradle 10 input/output port 18 is adapted for communication with the wireless primary communication device 40 and with the processing circuit 38. The I/O Port 18 optionally comprises a physical connection, or may comprise a wireless connection, or both.

Optionally, the cradle 10 includes a processor 38. The processor may be used alone or in combination with other processors, such as the internal processor of the wireless primary communications device 48. The form of processor may be of any type consistent with the inventions herein. The cradle 10 further includes memory 26, 28, 34. The memory serves to optionally store data indicative of the identification 26 of the cradle 10 application. Further, the memory may store data indicative of cradle identification information 28. The cradle 10 optionally includes a power source 32, such as a battery, or may utilize another power source, such as the power source 54 of the wireless primary communications device.

The cradle 10 additionally optionally includes an output 22 for interfacing with the body. One such output may provide radiation 24 to the body. Phototherapy may be provided to the body via the radiation. In another application, the output 22 device may transmit to the body information to control an implant within the body, such as a pacemaker or implanted pump. Optionally. a digital to analog (D/A) converter 20 is coupled to output 24.

The cradle 10 may additionally optionally include auxiliary sensors 30. One such sensor may be a temperature sensor. Optionally, an auxiliary external communication circuit 22 may be provided with the cradle 10.

As to the wireless primary communication device 40, it includes an internal processor 48. The form of processor may be of any type consistent with the inventions herein. The wireless primary communications device further includes memory 56. The memory serves to optionally store data indicative of the identification of cradle 10 application. Further, the memory may store data indicative of cradle 10 identification information. The wireless primary communications device preferably includes it's own power source 54, most commonly being a battery.

The wireless primary communications device preferably includes a housing 44 and a display 42 adjacent at least a portion of the housing 44. The display of the primary wireless communication devices is preferably flat. Optionally it may comprise a touch screen, such as an LCD touch screen. The display also may be a flexible display. 3-dimensional displays may also be utilized. A wireless external communication circuit 50 is included. It further includes a communication input/output port 46 adapted for communication with the cradle 10. The I/O Port optionally comprises a physical connection, or may comprise a wireless connection, or both.

Preferably, an attachment mechanism is provided to couple the wireless primary communications device and the cradle 10. The attachment mechanism may be a latch, such as a mechanical latch. Preferably, the attachment mechanism is releasable.

In yet another aspect, apparatus and methods include a cradle 10 for a modular system for mobile medical instrumentation for use with a body, the cradle 10 being adapted to interface with a wireless primary communication device. The cradle 10 preferably includes an input sensor 14 for receiving input from the body, the input sensor generating an output signal corresponding to the input, a processing circuit for receiving and processing the input sensor output signal, and a cradle 10 input/output port adapted for communication with the wireless primary communication device and with the processing circuit.

### Embodiment 1

### Electrocardiogram

A first embodiment and example of the invention is the implementation of a portable electrocardiograph. This application allows the health care provider to measure directly the electrocardiogram by placing the electrodes directly on the skin of the patient, avoiding the use of skin adhesives that have the potential to damage the skin; this feature is particularly crucial in premature and newborn babies, as the peeling of the electrodes damages the delicate skin of the infant. Furthermore, in addition to being inexpensive and portable, this embodiment of the invention is completely wireless, making it a one-piece handheld device without cables or added pieces. This feature is crucial in emergency situations, where rapid measurements are critical.

The electrode distribution is understood with particular reference to FIGS. 2 A, 2B, 3, 4 A, B and C, 5 and 6.

In classic, prior art, bipolar electrocardiogram recording, leads I, II, and III define a triangle known as Einthoven's triangle (shown in FIG. 2A). Lead I is measured by placing the negative electrode on the right arm and the positive electrode on the left arm; lead I forms a horizontal lead corresponding to the first side of the Einthoven's triangle. Lead II is measured by placing the negative electrode on the right arm and the positive electrode on the left leg; lead II forms a diagonal lead corresponding to the second side of the Einthoven's triangle. Lead III is measured by placing the negative electrode on the left arm and the positive electrode on the left leg; lead III forms a diagonal lead corresponding to the third side of the Einthoven's triangle.

A crucial component in the development of the device of the invention in this example is the fact that the Einthoven's triangle can be collapsed to a minimum of 3-4 cm side triangle (Human++: From technology to emerging health monitoring concepts. Penders, J. et al. 5th International Summer School Symposium on Medical Devices and Biosensors, 2008. pp 94-98 ISBN: 978-1-4244-2252-4). EKG signals have successfully been measured where the distance between electrodes has been on the order of 1 cm, and again on the order of 2 cm.

The present embodiment of the invention has three electrodes fixed on the back of the cradle 10 housing the cellular telephone. These electrodes are positioned in a way that they form a triangle with smaller dimensions than the previously described Einthoven's triangle (shown in FIG. 2 B). The device is placed and held by the operator on the skin of the chest of the patient in an area corresponding to the frontal projection of the heart of the patient. For proper operation, the orientation of the device will be such that two electrodes will be placed approximately parallel to an imaginary line described by the shoulders of the patient and the last electrode will be placed in caudal direction relative to the first two electrodes in an orientation similar to the one shown in FIG. 2 B.

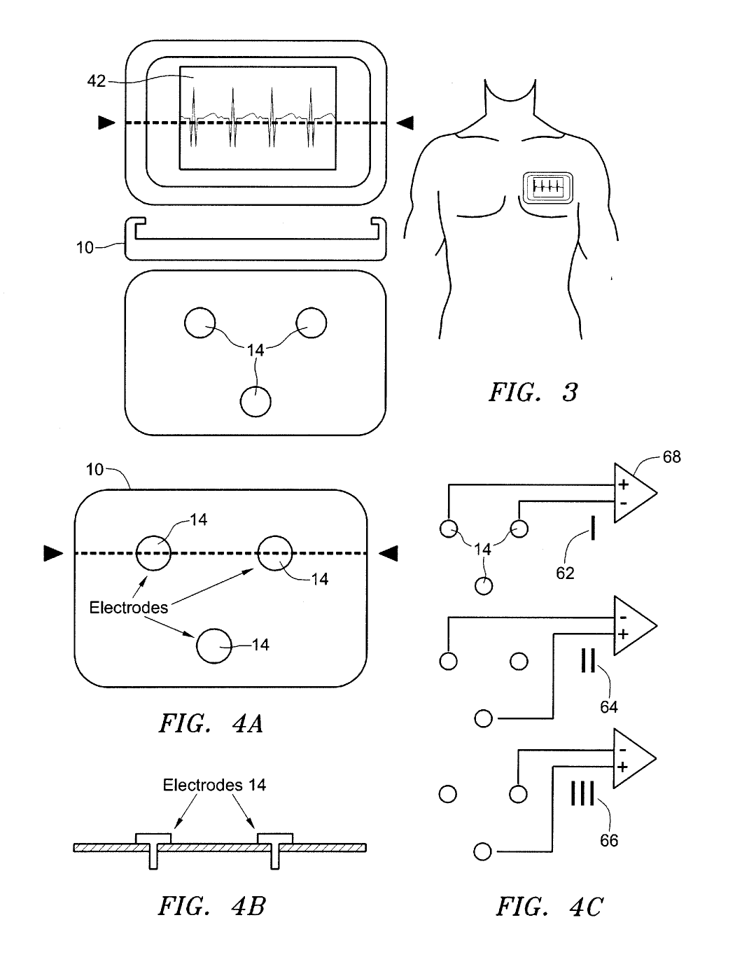

FIG. 3 is a general configuration of the device of the invention housing an electrocardiograph (ECG). The left top panel shows the device in operation with the cellular telephone 40 inside the cradle 10. The two triangular markers show the level of the cross section shown in the middle left panel of cradle 10 in cross-section. The middle left panel shows the cross section of the cradle without the cellular telephone inside. The lower left panel shows the back side of the cradle with the three electrodes 14. The right panel shows the device in operation and its placement on the chest of the patient. The electrocardiogram is directly shown on the screen 42 of the cellular telephone 40.

Configuration of the cradle 10, location of the electrodes and usage: FIG. 3 shows the general configuration of this embodiment. On the left top panel is shown the device in operation and the cellular telephone held by the cradle 10. The two triangular markers show the level of the cross-sectional projection shown in the middle left panel. The middle left panel shows the configuration of the cradle 10 only. The void space (white) represents the space occupied by the cellular telephone. Inside the cradle 10 there is a docking electronic connector to provide direct access to the input/output and power/charging ports of the cellular telephone. This docking connector will be used to connect the electronics of the cradle 10 with the cellular telephone. The lower left panel of FIG. 3 shows the back side of the cradle 10. The three circles represent the electrodes 14. The right panel of FIG. 3 shows the device in operation. The electrodes 14 in contact with the skin of the patient allow the device to detect the bioelectrical signal of the electrocardiogram. The signal is directly displayed on the screen of the cellular telephone.

Amplifier circuit and lead selection. FIGS. 4 A, B and C show the connectivity of the electrodes to the amplifier circuitry. FIG. 4 A shows the back side of the cradle 10 and the relative position of the electrodes 14. The triangular markers show the level of the view depicted in FIG. 4 B, and shows a cross sectional view across two electrodes 14. The upper part of the depiction in FIG. 4 B corresponds to the surface in contact with the skin of the patient during operation. With reference to FIG. 4 C, the electrodes' body reaches the inner side of the cradle 10 to allow connectivity to the electronics of the device, including the differential amplifier. The differential amplifier circuit is housed in the cradle 10 of the cellular telephone and it is used to determine the potential differences between the selected pair of electrodes. One of the electrodes feeds the positive (+) input of the differential amplifier and the other will feed the negative (−) input of the differential amplifier. The inputs for the operational amplifiers 68 are shown with the same polarity as described in FIGS. 2 A and B, given that the device in FIG. 4A is shown in plan view toward the electrodes of the cradle 10, whereas in use (in FIG. 2B), the device of FIG. 4 A would be oriented with the electrodes 14 oriented toward the patient. The electrodes are connected to a lead selector. For bipolar leads, when the lead selector is set to I (number one with roman numerals), the electrode on the right side of the patient will connect to the negative input and the left electrode will connect to the positive connector of the amplifier circuit, as shown in the upper panel of FIG. 4 C. When the lead selector is set to II, the right electrode will connect to the negative and caudal electrode will connect to the positive connector of the amplifier circuit, as shown on the middle panel of FIG. 4 C. When the lead selector is set to III, the left electrode will connect to the negative and the caudal electrode will connect to the positive connector of the amplifier circuit, as shown on the lower panel of FIG. 4 C.

Cell Phone Connectivity. This embodiment allows the user to send the electrocardiogram and the patient information either to a central computer system, for storage and future analysis, and/or to another cellular telephone.

### Embodiment 2

### Low Power Transmitter/Receptor of Biomedical Signals

In a second embodiment, the device of the invention is used to collect, store, display, relay, and transmit biomedical signals. This application allows the implementation of low-power, short range transmissions carrying the information of a biomedical signal to a cellular telephone.

In this embodiment, the cradle 10, described in detail in embodiment 1, houses a low power transmitter/receptor. One of the advantages of this embodiment is the reduction in the power of the radio signals applied to the patient.

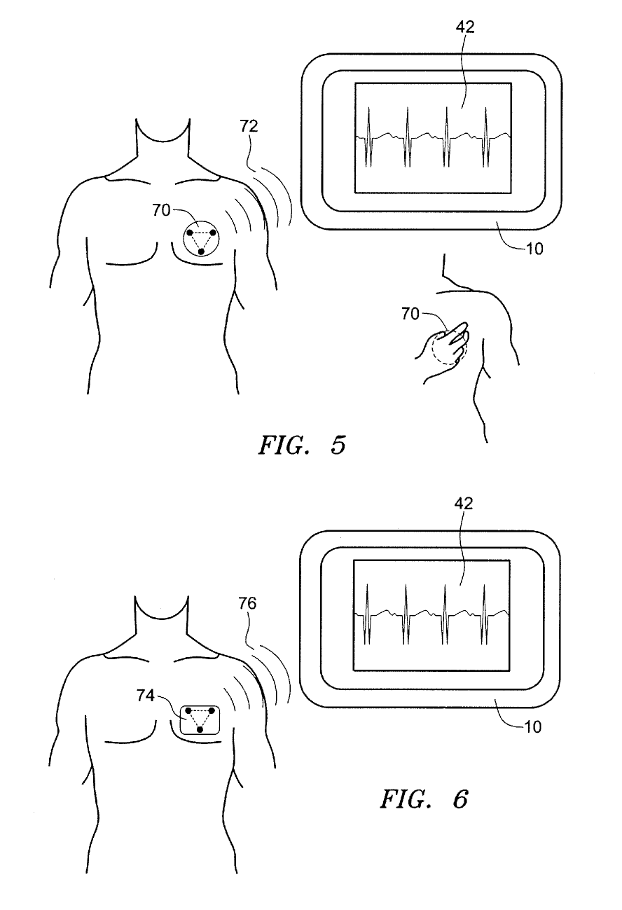

The first example of this embodiment is presented in FIG. 5. The electrode configuration to detect the electrocardiogram is similar to the description of embodiment 1. The electrodes are located in a handheld device, containing the amplifier circuit and a low power emitter. The left panel of FIG. 5 shows the example of a portable electrocardiogram in operation. The electrode configuration is similar to the configuration depicted in FIG. 2 B. The upper right panel of FIG. 5 shows a cellular device in its cradle 10. The cradle 10 contains the radio receiver and a docking connector that uses the input/output port to enter the data into the cellular telephone. The cellular telephone 40 displays 42 the biomedical signal on the screen. The phone is capable of storing, transmitting, and receiving data to and from other cellular telephones or a central computer system. The lower right panel depicts the positioning of the handheld device on the chest of a patient. Due to its small size, the patient or an operator can position the device.

A second example of this embodiment is presented in FIG. 6. In this example the electrodes are attached to an adhesive patch. The adhesive patch contains the amplifier circuit and a low power emitter. As in example 1 of this embodiment, the cradle 10 contains the necessary electronics for data reception and input to the cellular telephone.

As shown in FIGS. 5 and 6, the detection portion 70, 74 of the cradle 10 may detect and transmit wireless data 72, 76 to the phone including the display 42. If the wireless data 72, 76 is transmitted in a format that can be received and used by the phone, e.g., Bluetooth, then the cradle 10 portion physically adjacent the phone is optional. In the event that the detection portion 70, 74 transmits wireless data 72, 76 in a first format that cannot be received by the phone as it operates in a second format, the cradle 10 may serve as a bridge device to perform a translation from the first format of wireless data to the second format of wireless data used by the phone. Preferably, the cradle 10 includes a receiver operating in a first wireless data format, a translator to convert a first wireless data format to a second wireless data format utilized by the wireless primary communication device, where the first wireless data format is incompatible with the second wireless data format, the cradle 10 providing the second wireless data format to the wireless primary communication device. By way of example, the first wireless data format may be the ZigBee communication protocol and the second wireless data format may be any form usable with cellular communications devices, e.g., Bluetooth, GSM and/or CDMA. The primary communications device may optionally operate the two or more wireless data formats. For example, the communications unit may utilize Bluetooth wireless data format to transmit between it and the primary communications device, and the primary communications device may further then use a cellular wireless communications standard, e.g., CDMA or GSM, to communicate with the cellular network.

### Embodiment 3

### Point-of-Care Clinical Laboratory Testing

In a third embodiment, the device of the invention is used to measure clinical laboratory variables by housing the necessary hardware in the cradle 10 and utilizing a removable cartridge where the biological sample is placed.

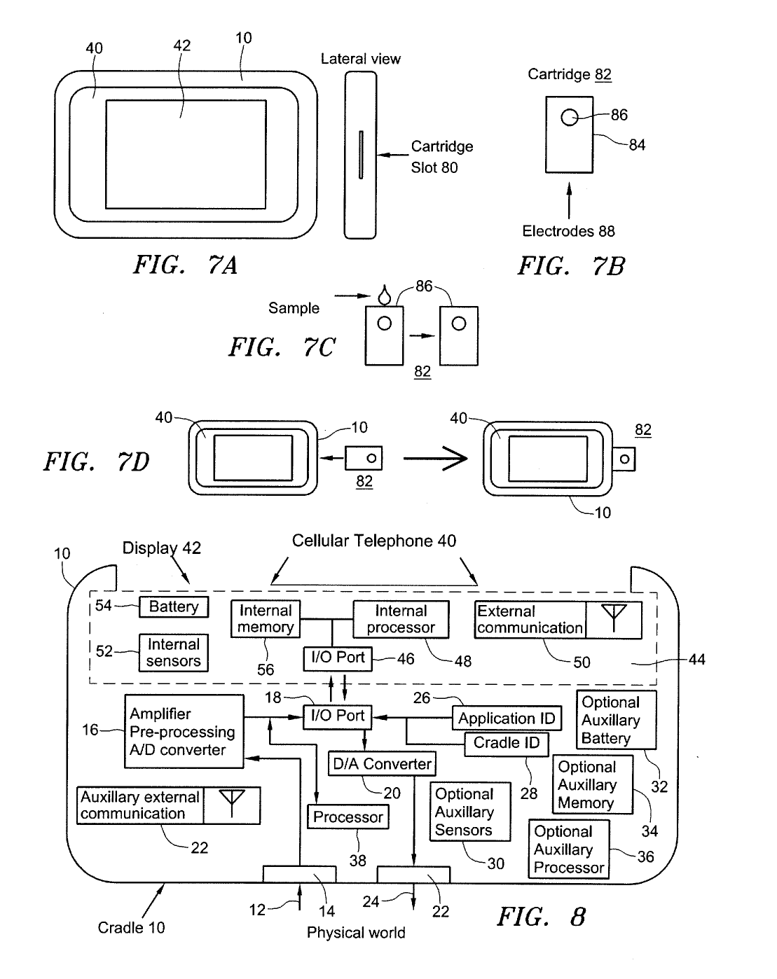

The first example of this embodiment corresponds to electrochemical methods, such as amperometric measurements using substrate specific enzyme-linked reactions, or voltmetric measurements. This example is presented in FIGS. 7 A through D. The cradle 10 has a lateral cartridge slot 80 for insertion of the cartridge carrying the biological specimen. FIG. 7 A shows the cellular telephone inside the cradle 10. The middle panel shows the lateral view of the cradle 10 showing the cartridge slot. FIGS. 7 B and C show depictions of the cartridge. The cartridge has an internal well to house fluid samples, the necessary reagents for the chemical reaction to take place, and the electric connections (see, electrodes 88 FIG. 7 B) between the reaction chamber and the contact electrodes. The cartridge electrodes connect to the circuitry of the cradle 10. FIG. 7 D depicts the placing of the biological sample in the cartridge and the placing of the cartridge in the cradle 10 for measurement.

The second example of this embodiment corresponds to optical methods to measure the concentration of the substance in the biological sample. For translucent samples the concentration can be measured using the well-known Beer-Lambert's law, in which the optical absorbance of the sample is a function of the concentration and the length of the path of the light beam. In this case, the length of the path corresponds to the thickness of the sample, i.e. the thickness of the cartridge, which is constant; thus, the differences in absorbance are due only to differences in concentration in two given samples. This example for this embodiment, utilizes a light source within the cradle 10 (for example, a light emitting diode) and a light intensity sensor (for example, a photoresistor).

### Embodiment 4

### Cell Counter

In a fourth embodiment, the device of the invention is used to measure the concentration of cells in a blood sample, using the Coulter's principle. In this embodiment a cartridge is used to handle a saline-diluted sample of blood. The impedance change across a narrow channel is measured while the blood sample is forced through. In an example of this embodiment, a microfluidics chip is used to handle the blood sample, house the channels, and provide the electric connectors to measure the impedance. This method allows measurement of the size and concentration of blood cells.

### Further Embodiments and Examples

In another embodiment, the device of the invention measures the absorbance of red and infrared wavelengths, necessary to evaluate the oxygenation level. In a first example of this embodiment the cradle 10 houses the electronics with the light emitter and detector. The cradle 10 has a void space where the finger or the earlobe is inserted for the measurement. In a second example of this embodiment, the light source and light sensor are placed on either a ring or a clip that can be placed on a finger or the earlobe of the patient. These devices can be connected to the cradle 10 of the device of the invention either with a wire or a wireless transmitter. In a third example of this embodiment, the light source and the light sensor are fixed on the surface, parallel to each other, and use the principle of reflective pulse oximetry (Independent Component Analysis Applied to Pulse Oximetry in the Estimation of the Arterial Oxygen Saturation (SpO₂)—a Comparative Study. Jensen, T. et al., 31st Annual International Conference of the IEEE EMBS Minneapolis, Minn., USA, Sep. 2-6, 2009, pp 4039-4044).

In another embodiment, the device of the invention performs spirometry measurements. The cradle 10 houses an air flow meter that connects to a mouth piece. The results of the flow measurements are plotted with respect to time to display volume-time curves and flow-volume loops. The device of the invention is capable of measuring physiologically relevant parameters such as: forced vital capacity, tidal volume, and total lung capacity.

In another embodiment, the cradle 10 of the device of the invention houses an infrared light source and an infrared detector to measure expired carbon dioxide concentration. The cradle 10 houses also a oxygen sensor probe. The device of the invention calculates the metabolic rate by measuring the total volume of the gas and the content of oxygen and carbon dioxide. This principle has been used by others to calculate the metabolic rate (U.S. Pat. No. 5,363,857 and U.S. Pat. No. 6,955,650). Nitric oxide may be measured via a nitric oxide sensor within a cradle 10, preferably via a nitric oxide electrode based gas sensor.

In another embodiment, the cradle 10 of the device of the invention houses an infrared thermography camera, connected to the video input of the cellular telephone. The practitioner uses this device to visualize find temperature differences on the body of the patient. This technology is valuable in finding breast cancer (infrared mammography), skin and sinus infections, and to detect areas of poor circulation in diabetes and other conditions.

In another embodiment, the cradle 10 of the device of the invention houses a plurality of Light Emitting Diodes (LEDs), or other source of light, used to provide phototherapy to the patient. These LEDs can be placed on the front surface, or the back of the cradle 10. These LEDs can provide phototherapy for the user, when using other capabilities of the cellular telephone. The cellular telephone has the capability to precisely quantify the dose of the phototherapy administered and report it to the practitioner.

All publications and patents cited in this specification are herein incorporated by reference as if each individual publication or patent application were specifically and individually indicated to be incorporated by reference. Although the foregoing invention has been described in some detail by way of illustration and example for purposes of clarity and understanding, it may be readily apparent to those of ordinary skill in the art in light of the teachings of this invention that certain changes and modifications may be made thereto without departing from the spirit or scope of the following claims.

1. A modular system for mobile medical instrumentation for use with a body, comprising:
a wireless primary communication device, the device comprising:
a housing,
a display adjacent at least a portion of the housing,
an internal processor,
a wireless external communication circuit,
a power source, and
a communication input/output port adapted for communication with a cradle,
at least one cradle, the cradle comprising:
an input sensor for receiving input from the body, the input sensor generating an output signal corresponding to the input,
a processing circuit, and
a cradle input/output port adapted for communication with the wireless primary communication device.

2. A modular system for mobile medical instrumentation for use with a body of claim 1 further including a processor for processing the input sensor output signal.

3. A modular system for mobile medical instrumentation for use with a body of claim 1 further including memory.

4. A modular system for mobile medical instrumentation for use with a body of claim 3 wherein the memory stores identification of the cradle application.

5. A modular system for mobile medical instrumentation for use with a body of claim 3 wherein the memory stores cradle identification information.

6. A modular system for mobile medical instrumentation for use with a body of claim 1 further including power source.

7. A modular system for mobile medical instrumentation for use with a body of claim 6 wherein the power source is a battery.

8. A modular system for mobile medical instrumentation for use with a body of claim 1 further including at least one temperature sensor.

9. A modular system for mobile medical instrumentation for use with a body of claim 1 further including an output for interfacing with the body.

10. A modular system for mobile medical instrumentation for use with a body of claim 9 wherein the output provides radiation to the body.

11. A modular system for mobile medical instrumentation for use with a body of claim 10 wherein the radiation is light therapy.

12. A modular system for mobile medical instrumentation for use with a body of claim 11 wherein the output information transmitted to the body provides information to an implant within the body.

13. A modular system for mobile medical instrumentation for use with a body of claim 12 wherein the implant is a pacemaker.

14. A modular system for mobile medical instrumentation for use with a body of claim 12 wherein the implant is an implanted pump.

15. A modular system for mobile medical instrumentation for use with a body of claim 9 further including a digital to analog (D/A) converter coupled to output.

16. A modular system for mobile medical instrumentation for use with a body of claim 1 further including auxiliary sensors.

17. A modular system for mobile medical instrumentation for use with a body of claim 1 further including auxiliary external communication circuit.

18. A modular system for mobile medical instrumentation for use with a body of claim 1 further including an attachment mechanism for wireless primary communications device.

19. A modular system for mobile medical instrumentation for use with a body of claim 18 wherein the attachment mechanism is a latch.

20. A modular system for mobile medical instrumentation for use with a body of claim 19 wherein the latch is mechanical.

21. A modular system for mobile medical instrumentation for use with a body of claim 18 wherein the attachment mechanism is releasable.

22. A modular system for mobile medical instrumentation for use with a body of claim 1 further including specific functional applications.

23. A modular system for mobile medical instrumentation for use with a body of claim 22 wherein the application takes an electrocardiogram.

24. A modular system for mobile medical instrumentation for use with a body of claim 23 wherein the input sensors are electrodes.

25. A modular system for mobile medical instrumentation for use with a body of claim 24 wherein there are 3 electrodes separated by 4 cm or less.

26. A modular system for mobile medical instrumentation for use with a body of claim 22 wherein the application includes point of care clinical lab testing.

27. A modular system for mobile medical instrumentation for use with a body of claim 26 wherein the point of care clinical lab testing includes electrochemical testing.

28. A modular system for mobile medical instrumentation for use with a body of claim 26 wherein the point of care clinical lab testing includes glucose testing.

29. A modular system for mobile medical instrumentation for use with a body of claim 22 wherein the application includes cell counting.

30. A modular system for mobile medical instrumentation for use with a body of claim 1 further including a respiration sensor.

31. A modular system for mobile medical instrumentation for use with a body of claim 30 wherein the respiration sensor detects CO₂.

32. A modular system for mobile medical instrumentation for use with a body of claim 30 wherein the respiration sensor detects O₂.

33. A modular system for mobile medical instrumentation for use with a body of claim 30 wherein the respiration sensor detects metabolic rate.

34. A modular system for mobile medical instrumentation for use with a body of claim 1 further including thermal sensor.

35. A modular system for mobile medical instrumentation for use with a body of claim 1 further including therapy device.

36. A modular system for mobile medical instrumentation for use with a body of claim 35 wherein the therapy device provides light for phototherapy.

37. A modular system for mobile medical instrumentation for use with a body of claim 1 wherein the cradle processing circuit includes an amplifier adapted to receive an input from the control system.

38. A modular system for mobile medical instrumentation for use with a body of claim 37 wherein the amplifier is a low noise amplifier.

39. A modular system for mobile medical instrumentation for use with a body of claim 37 further including the digital to analog converter disposed between the control system and the amplifier.

40. A modular system for mobile medical instrumentation for use with a body of claim 39 further including the digital signal processor between the control system and the digital to analog converter.

41. A modular system for mobile medical instrumentation for use with a body of claim 40 wherein an analog to digital converter is coupled to the amplifier.

42. A modular system for mobile medical instrumentation for use with a body of claim 40 wherein a digital signal processor coupled to the digital to analog converter.

43. A modular system for mobile medical instrumentation for use with a body of claim 1 wherein the input system detects biopotentials.

44. A modular system for mobile medical instrumentation for use with a body of claim 43 wherein the biopotential is an electromyogram (EMG).

45. A modular system for mobile medical instrumentation for use with a body of claim 43 wherein the biopotential is an electrocardiogram (ECG).

46. A modular system for mobile medical instrumentation for use with a body of claim 43 wherein the biopotential detects tissue fluid retention.

47. A modular system for mobile medical instrumentation for use with a body of claim 1 wherein input/output port comprises a physical connection.

48. A modular system for mobile medical instrumentation for use with a body of claim 1 wherein the input/output port comprises a wireless connection.

49. A modular system for mobile medical instrumentation for use with a body of claim 1 wherein the primary wireless device is a cellular phone.

50. A modular system for mobile medical instrumentation for use with a body of claim 1 wherein the primary wireless device is a PDA.

51. A modular system for mobile medical instrumentation for use with a body of claim 1 wherein the display is a flat panel display.

52. A modular system for mobile medical instrumentation for use with a body of claim 1 wherein the display is a touch screen.

53. A modular system for mobile medical instrumentation for use with a body of claim 1 wherein the display is a 3 dimensional display.

54. A modular system for mobile medical instrumentation for use with a body of claim 1 wherein the display is a LCD.

55. A modular system for mobile medical instrumentation for use with a body of claim 1 wherein the display is plasma display.

56. A modular system for mobile medical instrumentation for use with a body of claim 1 wherein the display is flexible display.

57. A modular system for mobile medical instrumentation for use with a body of claim 1 wherein the cradle further includes a receiver operating in a first wireless data format, a translator to convert a first wireless data format to a second wireless data format utilized by the wireless primary communication device, where the first wireless data format is incompatible with the second wireless data format, the cradle providing the second wireless data format to the wireless primary communication device.

58. A cradle for a modular system for mobile medical instrumentation for use with a body, the cradle being adapted to interface with a wireless primary communication device, the comprising:
a plurality of input electrodes for receiving input from the body, the electrodes providing an output signal corresponding to the input,
a processing circuit for receiving and processing the input sensor output signal, and
a cradle input/output port adapted for communication with the wireless primary communication device and with the processing circuit.

59. A cradle for a modular system for mobile medical instrumentation for use with a body, adapted to measure the electrocardiogram of a patient, the cradle being adapted to interface with a wireless primary communication device, the wireless primary communications device having a face surface of length L and width W, comprising:
an input sensor for receiving input from the body, the input sensor including multiple electrodes, the electrodes being located within the dimensions L and W, the input sensor generating an output signal corresponding to the input,
a processing circuit for receiving and processing the input sensor output signal, and
a cradle input/output port adapted for communication with the wireless primary communication device and with the processing circuit.

60. The cradle for a modular system for mobile medical instrumentation for use with a body of claim 59 wherein L and W are 4 centimeters or less.

61. The cradle for a modular system for mobile medical instrumentation for use with a body of claim 59 wherein L and W are 3 centimeters or less.

62. The cradle for a modular system for mobile medical instrumentation for use with a body of claim 59 wherein 3 electrodes are adapted to measure EKG potentials.

63. The cradle for a modular system for mobile medical instrumentation for use with a body of claim 62 wherein the 3 electrodes are arranged in an Einthoven triangular arrangement.

64. A modular multi-purpose system for medical instrumentation for use with a body, adapted for use with a wireless primary communication device having a housing, a display adjacent at least a portion of the housing, an internal processor, a wireless external communication circuit, a power source, and a communication input/output port adapted for communication with multiple cradles, comprising
a first cradle, the first cradle directed to a first medical use comprising:
an input sensor for receiving input from the body associated with the first medical use, the input sensor generating an output signal corresponding to the input,
a processing circuit for receiving and processing the input sensor output signal, and
a cradle input/output port adapted for communication with the wireless primary communication device and with the processing circuit, and
a second cradle, the second cradle directed to a second medical use, the second medical use being different than the first medical use, comprising:
an input sensor for receiving input from the body associated with the second medical use, the input sensor generating an output signal corresponding to the input,
a processing circuit for receiving and processing the input sensor output signal, and
a cradle input/output port adapted for communication with the wireless primary communication device and with the processing circuit.

65. A communications unit for mobile medical communication, for use with a first medical device adapted for use with a body and for wireless communication operating in a first wireless data format, and for use with a primary communication device, the primary communication device operating in a second wireless data format, the first wireless data format being incompatible with the second wireless data format, comprising:
a first receiver adapted to receive communications from the first medical device in a first wireless data format,
a translator to convert a first wireless data format to a second wireless data format utilized by the wireless primary communication device, and
a transmitter to convert the first wireless data format to the second wireless data format, and
a transmitter to send the converted second wireless data format to the primary communication device.
